(19) United States
(12) Patent Application Publication
(10) Pub. No.: US 2015/0306566 A1
(43) Pub. Date: Oct. 29, 2015

(54) SORPTION MATERIAL FOR THE SORPTION OF GAS MOLECULES, IN PARTICULAR CO2, IN MINIMALLY INVASIVE SURGICAL PROCEDURES

(71) Applicant: Cedar Advanced Technology Group Ltd., Zug (CH)

(72) Inventor: Kamal FARHA, Pleasantville, NY (US)

(21) Appl. No.: 14/647,384

(22) PCT Filed: Nov. 26, 2013
(86) PCT No.: PCT/CH2013/000203

§ 371 Date: May 26, 2015

(30) Foreign Application Priority Data
Nov. 30, 2012 (GB) 1221618.0

(51) Int. Cl. B01J 20/22; B01J 20/28; A61B 17/00; B01J 20/16

(52) U.S. Cl. CPC B01J 20/226; B01J 20/16; B01J 20/2808; B01J 20/28016; B01J 20/28033; A61B 17/00234; A61B 2017/0023; A61B 2017/00287; A61M 13/003

(57) ABSTRACT

The present invention relates to a sorption material for the sorption of gas molecules in a cavity of the body in minimally invasive surgical procedures. The sorption material comprises a zeolite, a Metallorganic Framework (MOF) or a mixture thereof.

The invention relates to a sorption material for the sorption of gas molecules, in particular carbon dioxide (CO₂), in a cavity of the human body in minimally invasive surgical procedures, such as laparoscopic procedures.

Laparoscopic surgery, also called bandaid surgery, or keyhole surgery, is a modern surgical technique, which involves a small incision in the abdomen or pelvis and the insufflation of the abdominal or pelvic area by a gas, so that the endoscope can view internal tissues without contacting said tissues.

The surgeon is allowed a magnified image of the abdominal contents displayed on TV monitors in order to have a better view of the area. During a laparoscopic procedure, approximately 15-20 litres of gas are insufflated into the abdomen, which has a capacity of about three litres. Thus, in order to allow performance of laparoscopic techniques, insufflation must reach a certain pressure starting from normal gas pressure present in the abdomen, which is normally less than 2-3 mmHg. For the laparoscopic procedure, pre-set pressures of 15 mmHg or less in the intra-abdominal space are considered safest to maintain pneumoperitoneum (presence of air or gas in the abdominal (peritoneal) cavity).

Gas delivery systems are generally composed of a containment cylinder, insufflator (gas throttling down pressure regulating unit), tubing, filter and abdominal entry device or port. After the procedure is completed, the surgeon releases the excess gas from the body by mechanical means or by wall suctions. However, this is often insufficient as several millilitres of gas are left in the body.

The gas used is generally CO₂, which is common to the human body and can be absorbed by tissue and removed by the respiratory system. It is also non-flammable, which is important because electrosurgical devices are commonly used in laparoscopic procedures. Also, the risk of gas embolism is lowest with CO₂.

Nevertheless, there are also other gases or gas mixtures, which are used in some cases. N₂O gas, which has a similar molecular weight as CO₂, is often used for patients with cardiac problems since CO₂ can induce hypercarbia. There are numerous advantages to this technique versus an open surgical procedure; such as a smaller incision leading to less post-operative pain, less haemorrhaging and reduced hospital stays for patients, thus reducing risk for potential infection or other complications.

However, a disadvantage to the technique is that patients may experience severe post-operative pain, independent of the incision and incision site. It has been suggested that this pain is associated with insufflating the abdominal area and in particular with incomplete removal of carbon dioxide from the body. There was found a correlation between the volume of residual gas and the severity of pain that patients experienced after laparoscopic procedures. Recent studies confirm that residual gas in the abdominal cavity plays a major role in the production of post-operative pain after laparoscopy.

Since the pain usually occurs in the upper abdomen, back or shoulders, involvement of phrenic nerve pressure is assumed. The phrenic nerve originates from the cervical nerves, which provide the motor supply to the diaphragm as well as other sensations. Without wanting to be bound by the theory, it is assumed that when gases are used to blow up the abdominal region, they tend to rise and will begin to push on the diaphragm and thus leading to phrenic nerve irritation. Phrenic nerve irritation will then affect other areas of the body, including the back and shoulders, as previously stated.

It has been shown that the pain can be reduced by repeated suction with use of a gas drain. However, after minor laparoscopic surgery, it is less cost-effective to reduce pain using an intraperitoneal gas drain than simple oral analgesia.

Effective materials and methods of removing specific gas components, inter alia of CO₂, have been developed for industrial purposes and are e.g. described by Wong and Bioletti in “Carbon Dioxide Separation Technologies”, Alberta Research Council. These materials and methods include physical solutions, cryogenic separation, membrane separation and chemical absorption.

In the industrial field of exhaust gas purification, pressure swing adsorption (PSA) is a common commercial process which utilizes pressure changes to promote the cyclic adsorption and desorption of the gas. Generally, a column packed with a highly porous reversible adsorbent, such as activated carbon or surface modified zeolites is employed.

Zeolites are aluminosilicate-based materials with porous structures that absorb a multitude of positive ions but also gas molecules such as CO₂. Thanks to their highly porous and consistent matrix, zeolites can be used as inorganic molecular sieve membranes to selectively separate molecules based on charge and size.

Industrial sorption units often use a bed of sorbent based on one or a mixture of zeolite types A, X and Y, in particular zeolite types 4A, 5A, or faujasite-type zeolite called zeolite 13X.

There has been on-going research in order to improve the sorption capacity and to optimize the material transfer properties of the zeolites used in industrial sorption processes.

U.S. Pat. No. 3,885,927, for instance, teaches that the adsorption of CO₂ may be effected on a zeolite X exchanged to more than 90% with barium, whereas EP 294 588 teaches the use of zeolite X preferably exchanged to 70% with strontium in order to carry out this purification.

U.S. Pat. No. 5,531,801 and EP 718 024 teach that it is possible to adsorb CO₂ very effectively by means of an X-type zeolite with an Si/Al ratio of less than 1.15 and preferably equal or very close to 1, called zeolite LSX (Low Silica X).

EP 1 062 022 shows that a very appreciable gain in efficiency may be obtained in respect of decarbonisation at low CO₂ partial pressures (of around 2 mbar) using LSX zeolites whose degree of sodium exchange (defined as the molar ratio of sodium ions to aluminium atoms in the tetrahedral position, the remainder being potassium) is at least 98%.

WO 00/01478 describes NaKLSX adsorbents in which the Si/Al ratio is between 0.9 and 1.1, the K⁺ ion content is less than 8%, the macroporous volume is greater than 0.4 cm³/g, containing small crystals (1-4 μm) that can be used for the decarbonization of gas streams. The use of such molecular sieves showed an increase in dynamic adsorptivity at room temperature in the case of low CO₂ partial pressures.

WO 2010/138080 discloses a metabolically inert gas absorber compound on the basis of zeolite, salt and a binding agent for the production of a licking element for ruminants. The zeolites in the licking element are used to bind undesirable gases, such as methane, which are produced in the digestive systems of ruminants and that otherwise would escape via the breath or the intestines.

GB 2 259 858 relates to a container, particularly a sachet, containing a natural or synthetic zeolite in the form of fine dust, powder, granules or crystals the sachet being air/gas/water permeable and preventing significant escape of the contents. The sachet, which can resemble a “tea bag”, may include double sided adhesive tape to allow it to be placed easily on the outer surface of a wound, an ulcer dressing or an incontinence pad, or on the inner surface of a stoma bag.

Apart from zeolites, Metallorganic Frameworks (MOFs) materials have been demonstrated to be effective both as a CO₂ adsorbent and as a catalyst for its chemical fixation (Yang D.-A. et al, Energy Environ. Sci., 2'12, 5, 6465). MOFs are a relatively new class of crystalline materials composed of organic and inorganic moieties in a 3-D arrangement having huge surface areas and pore volumes.

Zeolitic Imidazolate frameworks (ZIFs) are members of the MOF family and are generally constructed by linking four-coordinated transition metals through imidazolate units to yield extended frameworks based on tetrahedral topologies. ZIFs often have topologies analogous to zeolite structures, having large pores and high affinity to carbon dioxide.

Whereas many ways have been found to remove gas molecules in industrial applications, such as the purification of exhaust gases, the need for effective and cost-effective means for removing the excess gas molecules, usually CO₂ molecules, from a body's cavity is still unmet.

The problem of the present invention is to provide a sorption material as an effective means for use in a minimally invasive surgical procedure for the sorption of gas molecules in a body's cavity.

The problem is solved by a sorption material according to claim 1 for the sorption of gas molecules in minimally invasive surgical procedures, the packaging according to claim 11 and the kit according to claim 14. Further preferred embodiments are subject of the dependent claims.

The sorption material of the present invention is for the sorption of gas molecules in a cavity of the body in minimally invasive surgical procedures. The sorption material comprises a zeolite, a Metallorganic Framework (MOF) or a mixture thereof. In other words, the sorption material is intended for use in minimally invasive surgical procedures for the sorption of gas molecule in a cavity of the body.

In the context of the present invention the term “minimally invasive surgery” includes all kinds of endoscopic surgical procedures, i.e. laparoscopic surgery, bariatic surgery, gynaecological surgery, endoscopic endocrine neck surgery, robotic surgery, vessel harvesting etc., during which a working space in the body is created by gas insufflation, in particular CO₂ insufflation.

The sorption material of the present invention allows for effective removal of gas molecules in or after minimally invasive surgical procedures, which reduces post-operative pain of the patients and leads to less pain medication prescribed. In addition, the costs can be lowered since the hospital stays are significantly reduced.

Thus, the sorption material of the present invention is for use in the reduction of post-operative pain after minimally invasive surgery. Throughout this application, the term “sorption” is used for a process where molecules are taken up by the surface (adsorption) or the volume (absorption) of a material. In other words, molecules of a substance A are taken up by the surface or the volume of a material B.

It was found that the sorption material of the present invention is stable and effective in the temperature range of 10 to 40° C. This makes the sorption material highly suitable for application in surgical procedures, which are carried out in this temperature range.

According to the present invention, the sorption material comprises a zeolite and/or a Metallorganic Framework (MOF), which have shown to be highly effective for the sorption of gas molecules, and in particular of those generally used in minimally invasive surgeries. Thanks to their environmentally friendly and generally non-toxic character, zeolites and MOFs are particularly well suited as sorption material for use in such surgical procedures.

According to a preferred embodiment, the sorption material allows for the sorption of gas molecules in an abdominal cavity of the body in laparoscopic procedures.

Since CO₂ is generally used in such minimally invasive surgical procedures, the sorption material of the present invention preferably allows for the sorption of CO₂ gas molecules in a cavity of the body. In this regard, the present invention is not limited to pure gases but also allows for the sorption of gas mixtures, e.g. for the sorption of CO₂ and carbon monoxide (CO). The effective removal of the latter will be explained further below.

In order to efficiently ad-/absorb specific gas molecules, the sorption material preferably has a sorption capacity of at least about 20 grams of the respective gas molecules per 100 grams of sorption material.

In a preferred embodiment, the sorption material comprises at least one of the zeolite types A, B, X, LSX (Low Silica X) and Y, in particular zeolites belonging to the group of faujasites (type Y, X, ALSX) or to the group of A-type zeolites (LTA). Zeolite types 4A, 5A, 13X, chabazite (e.g. SSZ-13) and NaKLSX are particularly preferred.

In another preferred embodiment the sorption material preferably comprises a MOF which belongs to the group of Zeolitic Imidazolate frameworks (ZIFs).

Due to the low production costs and their ability to remain stable over the temperature range used during operations, ZIFs are preferred sorption materials for use according to the present invention.

As mentioned before, minimally invasive surgeries generally use CO₂ gas, which is inflated into a body's cavity in order to create a sufficient working space for surgical instruments and viewing space for optics.

Therefore it is preferred that the sorption material of the present invention comprises Mg-MOF, in particular Mg-MOF-74, since Mg-MOFs have the beneficial properties of being able to take up 5-10% of its own weight in CO₂ and of tolerating temperature shifts while maintaining their properties. Further preferred MOFs include MOF-5, IRMOF-1, MOF-177, MIL-53, MIL-100 and MIL-101.

Particularly preferred ZIF types are ZIF-8, ZIF-69, ZIF-78, ZIF-95 and ZIF-100. Their additional beneficial characteristics of having a low density, high surface area and robust structure, they allow a particularly effective sorption of gas molecules, and in particular of CO₂. ZIF-100, for instance, has been found capable of storing 28 litres CO₂ per litre of material at standard temperature and pressure. An extraordinary sorption capacity of 82.7 litre CO₂ per litre ZIF material was reported for ZIF-69 at a low pressure of 1 atm at 25° C. (Banerjee, R. et al, J. Am. Chem. Soc. 131 (11), 3875-3877, 2009).

In the context of the present invention, the above mentioned preferred Zeolite or MOF types include both, their non-modified and modified types. For example, the term “ZIF-8” encompasses non-modified ZIF-8 but also modified types such as e.g. amino-modified ZIF-8-NH₂ and ZIF-8-(NH₂)₂.

Although CO₂ is the gas which is most frequently used in minimally invasive surgeries, the sorption material of the present invention allows also for the sorption of other gas molecules in a cavity of the body in such surgical procedures. These gases involve air, oxygen, nitrous oxide (N₂O), argon, helium and mixtures thereof, which are sporadically used in minimally invasive surgical procedures.

For absorption of helium, for instance, the zeolite-NaA, has shown to be particularly effective as a helium sorption material. In case of Argon, a sorption material comprising zeolite-ZSM-5 is preferred.

In this context, the present invention also provides a sorption material for the removal of gaseous by-products formed during minimally invasive surgical procedures. This will be further explained below:

Modern minimally invasive surgical procedures frequently involve electrosurgery or electrocautery and also lasers have become increasingly popular. However, the use of these devices tends to create surgical smoke in the working space due to burning of tissue. Known toxic materials, which may be created as by-products resulting from pyrolysis of protein and lipids include Acroloin, Acetonitrile, Acrylonitrile, Acetylene, Alkyl Benzenes, Benzene, Butadiene, Butene, Carbon monoxice, Creosols, Ethane, Ethene, Ethylene, Formaldehyde, Free Radicals, Hydrogen cyanide, Isobutene, Methane, Phenol, PAH's, Propene, Propylene, Pysidene, Pyrrole, Styrene, Toluene and Xylene. On a toxicological basis, tissue combustion within the closed abdomen at laparoscopy is an iatrogenic smoke-poisoning incident. Smoke evacuation systems which use a discharge limb are commonly used to remove the smoke from the surgical site, so that a surgeon can see what he or she is doing, and so that this potentially harmful material does not remain within the body cavity post-surgery.

In this regard, the present invention provides a much simpler and cost-saving method for the removal of potentially harmful gaseous substances. In particular, the removal of carbon monoxide (CO) is of high importance since CO is one of the most lethal products listed above. Furthermore, elevated CO emissions, so-called “smoke” are common in the laparoscopic situation as it is often caused by combustion processes that occur in low oxygen environments.

By the present invention, the removal of CO during surgery from within an insufflated surgical cavity can be quickly and effectively achieved by using a sorption material described herein. Specifically, zeolite-5A was shown to have the highest adsorption capacity for CO at ambient temperature (25° C.) and ambient pressure (108 kPa), whereas MOF-177 presented to be particularly effective at lower temperatures. In contrast to other known smoke evacuation systems, sorption of CO can be effected much quicker and easier by the use of the sorption material of the present invention, thus less time is required to return to preoperative levels.

As has been mentioned above, the sorption material is not limited to the sorption of only one sort of gas molecules but may also be chosen such that it allows for the sorption of gaseous mixtures, in particular of CO₂ and CO.

Preferably, the sorption material has pores which have an average pore diameter of less than 1 nm, preferably less than 0.7 nm, most preferably equal to or less than or 0.5 nm. This pore size has been proven effective to remove excess gas molecules from a closed compartment. Specifically, zeolites of 4A or 5A have shown to be highly effective.

It is further preferred that the sorption material has a pore volume of at least 0.01 cm³/g, more preferably at least 0.04 cm³/g, most preferably at least 0.15 cm³/g. This pore volume allows for a high uptake or sorption capacity for small gas molecules as those that are generally used in minimally invasive surgeries.

Methods for measuring the pore volume are known in the art and can be conducted e.g. by ASTM D4404-10, which is a standard test method for the determination of pore volume and pore volume distribution of soil and rock by Mercury Intrusion Porosimetry.

In order to achieve a high sorption capacity, the sorption material has preferably an active surface of at least about 5 m²/g, more preferably of at least about 15 m²/g, most preferably of at least about 30 m²/g.

Preferably, the sorption material is in form of a solid body or in powder form. A solid form is for example a cube, a cylinder or the like, which is suitable for being held by an at least partly insertable surgical instrument.

In case of the sorption material being in powder form, the material may be formed into granules, beads or pellets for ease in handling and transportation. The use of a paste-like composition is also an option. Such a paste-like composition has the advantage that it can be easily applied on a surgical tool without spilling.

For allowing the sorption material to be shaped into beads, pellets or similar structures, the sorption material may, besides the constituents mentioned above, also comprise additives such as fillers, antioxidants, binders, stabilizers, hardeners and the like. Methods for forming beads or pellets are well known in the art.

Examples of binders which may be used in the sorption material according to the present invention are e.g. clays that can be zeolitized, such as kaolin, metakaolin and halloysite, by themselves or as a blend.

In case of a powder, it is further preferred that the powder has particles with an average size of less than or equal to 10 μm, preferably less than or equal to 5 μm, most preferably less than or equal to 2 μm. In some embodiments, the average grain size of the powder particles is as small as 10 to 100 nm.

Various methods for measuring the particle size are known to a person skilled in the art, such as Sieve analysis, Malvern, Low-Angle-Laser-Light-Scattering, Dynamic Light Scattering, Laser-Diffraction, Spatial Filter Velocimetry, Image Analysis or Micromeritics, for instance.

In a further preferred embodiment the sorption material comprises additional nanoporous sorbents, such as COFs and silicon nanotubes.

It is understood that the sorption material of the present invention is preferably non-toxic or harmful for the human body as it is for the use in surgical procedures. In another aspect, the present invention further relates to a packaging system containing the sorption material.

When using the sorption material of the present invention, the material must be prevented from spilling inside the body's cavity, which could lead to contamination and risk of infection. This can be avoided by the gas-permeable packaging of the present invention, which contains the sorption material of the present invention and allows for an easy and fast insertion/removal of the sorption material into/from the incision site of a patient.

The gas-permeable packaging is made of a material that allows the penetration of gas molecules, and in particular CO₂ molecules, such that they can be ad- or absorbed by the sorption material.

Preferably, the gas-permeable packaging of the present invention is sterile in order to avoid contamination of the incision site. The packaging may be sterilized, for example, with gamma radiation.

It is further preferred that the material of the packaging has pores, having an average diameter of 1 to 50 nm, preferably of 1 to 30 nm, more preferably 2 to 10 nm. This pore size allows for the penetration of small gas molecules such as CO₂ but prevents the packed material from spilling from the packaging. The packaging material may also have a graded pore structure.

The gas-permeable packaging is preferably made of a material such as woven fibrous materials, non-woven fibrous materials, membranes, puffs, sponges and mixtures thereof. Fibres used to make such woven or non-woven fibrous materials may include aramids, acrylics, cellulose, polyester, chemically modified cellulose fibres and mixtures thereof. In a preferred embodiment, the gas-permeable packaging of the present invention is in the form of an envelope or a bag. Further preferred forms are more or less rigid structures in any form, three-dimensional shape and/or size suitable for packaging the sorption material. The packaging may therefore also be in form of a container, box, can or basket, which has a rectangular, cylindrical, spherical or polygonal shape.

That way, the packaging comprising the sorption material of the present invention may be held by tweezers or by means of another surgical instrument for being inserted into the body's cavity.

In particular if the sorption material is used for sorption within an insufflated surgical cavity, the packaging is preferably delivered and retrieved through a thin tube, also called trocar. This way, the packaging can be conveniently and accurately introduced into the surgical cavity to a desired location.

In a further preferred embodiment, the gas-permeable packaging itself is in the form of a thin cylindrical tube, which is made from a material such as e.g. surgical grade steel alloys or PET based plastic, and the sorption material is located inside, in an end portion of the tube. For the use of the sorption material during laparoscopic procedures according to the present invention, the sorption material is attached to an insertion aid, such as a robotic surgical apparatus, inserted through a small incision into the incision site, e.g. into the upper diaphragm, allowed to ad- or absorb gas molecules present in the body's cavity and is subsequently removed through the incision.

The gas-permeable packaging according to the present invention therefore allows for an easy and fast insertion and safe removal of the sorption material into/from the incision site of a patient.

The packaging also allows for delivering specific amounts of the sorption material.

The gas-permeable packaging containing the sorption material is preferably further contained within an outer gas-tight packaging to prevent the sorption material to take up gas molecules present in the air before its use according to the present invention. Further, since some sorption materials require a certain water content or humidity, the gas-tight packaging further prevents the loss of humidity.

In a preferred embodiment the sorption material is contained in a plastic material, which comprises antimicrobial elements like silver or silver nanoparticles to minimize the risk of infection due to the sorption material and/or the surgical items used for inserting the sorption material into the incision site during the surgical procedure. Such an embodiment has the clear advantage that is scavenge the carbon dioxide and sterilizes the wound in order to minimize the infection risk. Said plastic material is made from an antimicrobial resin, that is a resin, such as an acrylic based multipolymer comprising silver or silver nanoparticles. In another aspect, the present invention further relates to a kit comprising a sorption material according to the present invention and/or a gas-permeable packaging as described above as well as a surgical device for use in surgical procedures. Said surgical device has holding means for holding the sorption material and therefore allows for inserting and removing the sorption material in/from an incision site.

Another aspect of the present invention relates to the use of a sorption material in the manufacture of a medicament for the sorption of gas molecules in a cavity of the body in minimally invasive surgical procedures, wherein the sorption material comprises a zeolite, a Metallorganic Framework (MOF) or a mixture thereof.

### EXAMPLES

### Synthesis of Compounds

Light yellow crystalline plates of ZIF-95 (framework composition: Zn(cbIM)₂) were isolated from the reaction of a mixture containing Zn(NO₃)₂.4H₂O, 5-chlorobenzimidazole (cbIM), N,N-dimethylformamide (DMF) and water at 120° C. (75.0% yield based on zinc).

Dark yellow cubic crystals of ZIF-100 (framework composition: Zn₂₀(cbIM)₃₉(OH)) were isolated from the reaction of a mixture containing Zn(O₃SCF₃)₂, 5-chlorobenzimidazole (cbIM), N,N-dimethylformamide (DMF) and water at 120° C. The added water was precisely controlled. ZIF-100 was obtained in a 70.5% yield. Precise control of water added and use of under the same conditions yields (70.5% yield).

For the fabrication of a Li₂CO₃/Al₂O₃ composite by spray drying and post-processing, 140 g of a powder consisting of 10% Al₂O₃ is put into 2 quartz boats, which are then loaded into a tube furnace. Under flowing air, the furnace is ramped 10° C./min to 500° C., held for 3 hours and is then cooled to room temperature. The resulting powder consists essentially of Li₂CO₃ and Al₂O₃.

### Example of Use

A probe comprising ZIF-95 in form of a microcrystalline powder was packed inside a gas-permeable bag and held between sterile tweezers. Upon completion of the laparoscopic procedure, a main part of the CO₂ gas inflated into an abdominal cavity was released through the incision site. Before closing the incision, the probe held by sterile tweezers was inserted into the incision site, allowed to ad-or absorb residual CO₂ gas and was subsequently removed.

1. Sorption material for the sorption of gas molecules in a cavity of the body in minimally invasive surgical procedures, wherein the sorption material comprises a zeolite, a Metallorganic Framework (MOF) or a mixture thereof.

2. Sorption material according to claim 1 for the sorption of gas molecules in an abdominal cavity of the body in laparoscopic procedures.

3. Sorption material according to claim 1, for the sorption of CO2 gas molecules.

4. The sorption material according to claim 1, wherein the zeolite comprises one or more of the zeolite types A, X, LSX and Y.

5. The sorption material according to claim 1, wherein the Metallorganic Framework comprises a Metallorganic Framework type which belongs to the group of Zeolitic Imidazolate Frameworks (ZIF).

6. The sorption material according to claim 1, wherein the Metallorganic Framework comprises at least one material selected from the group of Mg-MOF-74 MOFs, MOF-5, IRMOF-1, MOF-177, MIL-53, MIL-100, MIL-101, ZIF-8, ZIF-69, ZIF-78, ZIF-95 and ZIF-100.

7. The sorption material according to claim 1, wherein it has pores which have an average pore diameter of less than 1 nm.

8. The sorption material according to claim 1, wherein it is in form of a fibrous, granular, pelletized or one-piece solid body.

9. The sorption material according to claim 1, wherein it is in powder or paste-like form.

10. The sorption material according to claim 9, wherein the powder has particles with an average size of less 10 than or equal to 7 pm.

11. Sorption material for use in a cavity of the body in minimally invasive surgical procedures to sorb gas molecules, wherein the sorption material comprises a 15 zeolite, a Metallorganic Framework (MOF) or a mixture thereof according to claim 2.

12. Gas-permeable packaging containing the sorption material according to claim 1.

13. Gas-permeable packaging according to claim 2, wherein the packaging is made of a material selected from the group consisting of woven fibrous materials, nonwoven fibrous materials, puff, sponges and mixtures thereof.

14. Gas-permeable packaging according to claim 12, wherein the material of the packaging has pores, having an average diameter of 1 to 50 nm.

15. Kit comprising a sorption material according to claim 1 and/or a gas-permeable packaging and a surgical device for use in surgical procedures, wherein the surgical 5 device has holding means for holding the sorption material.
